(19) United States
(12) Patent Application Publication
(10) Pub. No.: US 2022/0354201 A1
(43) Pub. Date: Nov. 10, 2022

(54) COSMETIC TREATMENT METHOD AND ASSEMBLY FOR PERFORMING SAID METHOD

(71) Applicant: L'OREAL, Paris (FR)

(72) Inventors: Henri SAMAIN, Chevilly LaRue (FR); Jean-Baptiste BLANC, Chevilly LaRue (FR); Franck Giron, Chevilly LaRue (FR); Leah Legoai, Wendywood (ZA)

(21) Appl. No.: 17/621,350

(22) PCT Filed: Jul. 29, 2020
(86) PCT No.: PCT/EP2020/071446

§ 371 Date: Dec. 21, 2021

(30) Foreign Application Priority Data
Jul. 31, 2019 (FR) FR1908749

(51) Int. Cl. A41G 5/00; A61K 8/02

(52) U.S. Cl. CPC A41G 5/008; A61K 8/027

(57) ABSTRACT

A cosmetic treatment method including the steps of (a) applying an adhesive to an area of the scalp, (b) applying fibres to said area covered with adhesive, belonging to a bunch of at least 50 entangled fibres, said bunch including fibres having, at least over part of their length, a radius of curvature of between 1 and 10 mm, preferably between 1 and 5 mm, the application taking place in such a way that for at least some of the fibres of the bunch, they only come into contact with the adhesive over part of their length.

### TECHNICAL FIELD

The present invention concerns cosmetic treatments of the scalp and more specifically those directed at alopecic areas.

### PRIOR ART

The curlier the hair, the smaller its diameter of curvature, and, since it stretches less under stress, it is more liable to break.

Creating or maintaining a hair care beauty treatment for curly hair requires significant effort. Thus, in order to save time, routines aim to achieve a hairstyle that will last for several days. These routines are stressful in terms of the hair because they involve the application of considerable mechanical forces to the hair, when braiding it tightly or adding extensions for example. With repeated application, these forces overload the intrinsic fragility of curly hair and in the long term can cause frontal and/or temporal alopecias.

When, in order to conceal these alopecias, the person resorts to new braids or extensions, this tends to sustain or even accelerate the phenomenon of hair breakage.

Furthermore, various techniques exist today for concealing or correcting alopecia:
hair implants that require surgical procedures;
hairpieces or wigs;
“pepper shaker” systems depositing millimetric particles which add bulk to the hairs still present in the thinning areas in order to make them more visible;
colouring the skin by application of a conventional makeup or colouring product or in a more invasive and permanent manner such as tattooing;
application of free fibres or fibres within a gel-type formula.

Apart from the invasive, expensive, surgical procedure that is difficult to put right if it is done poorly, it is difficult to have a form of alopecia correction that is undetectable both close up and from afar, that is to say that accurately reproduces the implantation and the density of human hair and does not expose the user to the risk that an outside observer may notice that the head of hair is not entirely natural.

A method of concealing alopecic areas is described in application WO 2017/021240, but its utilisation is limited to straight hair.

WO 2019/143 132 discloses a strand which has a shape extending in a longitudinal direction that includes one kind of filament having an internalized gradient-length effect by a spiral rotation twist and having a fine texture caused by irregular autogenous heat shrinkage.

Wigs and hairpieces offer certain advantages but lead to a fear of displacement over the course of the day, and hence a risk that their presence will be detected by outside observers. Moreover, for the people using them, they are thought of as a prosthesis, which for them has a negative and off-putting effect. In addition, they are not suitable for most cases of alopecia, in particular when the scalp is still partially covered with hair and when hairstyles are short. One solution is to attach the wig or hairpiece by means of ties which are supported by the hair, in particular by weaving or stitching them to the natural hair. However, this approach takes a very long time and requires the dexterity of hairdressers, making it unsuitable when regular hair washing is desired. In addition, these fixings may cause breakage problems in the long term. Finally, given the cost of wigs and hairpieces, the user will be likely to reuse them. Here too, washing and drying these wigs and hairpieces is regarded as a negative and off-putting experience. Thus, this solution is unsuccessful.

### PRESENTATION OF THE INVENTION

There is thus a need, not met to date, to benefit from an easy-to-use method for concealing alopecia in people with curly hair, in a manner that respects the natural hair, with a result that is virtually undetectable.

### SUMMARY OF THE INVENTION

The invention aims to meet this need and does so by virtue of a cosmetic treatment method comprising the steps consisting of:

(a) applying an adhesive to an area of the scalp,
(b) applying fibres to said area covered with adhesive, belonging to a bunch of at least 50 entangled fibres, comprising fibres having, at least over part of their length, a radius of curvature of preferably between 1 and 10 mm, the application taking place in such a way that for at least some of the fibres of the bunch, they only come into contact with the adhesive over part of their length. Preferably, for at least half the fibres of the bunch, better still at least 75% of the fibres of the bunch, even better at least 90%, they only come into contact with the adhesive over at most 20% of their length, better still at most 10% of their length. “Entangled fibres” are understood to be fibres that are interconnected by virtue of their shapes and the mechanical interactions generated by their intertwining, the intertwined fibres thus being held without the presence of glue between the entangled fibres or of bonds between the entangled fibres.

The invention makes it possible to achieve a result that is perceived as natural and undetectable.

In particular, the invention makes it possible to obtain relatively easily, if desired:
a density of fibres close to the natural density,
a distribution of fibres close to the natural distribution,
a location limited to only the areas to be treated,
the possibility of treating glabrous areas and thinning areas.

The treatment can be readily carried out in a few minutes, every day if required, without outside assistance. The fibres can be applied manually to said area, in particular by tapping the area covered with adhesive with the bunch held between two fingers. Where appropriate, the deposited fibres can be rubbed after being brought into contact with the adhesive. In particular, the area covered with adhesive and with the fibres deposited thereon can be swept with the fingers or hand.

Preferably, some of the fibres of the bunch remain attached to the fibres already deposited, by entanglement therewith, without coming into contact with the adhesive.

Preferably, the fibres are applied in such a way that SL is between 6 and 12%, where SL is the ratio between, firstly, the sum of the lengths of fibres deposited with a portion thereof in contact with the adhesive and, secondly, the sum of the lengths of all the fibres of the bunch. The method may include the application of fibres from at least two bunches to said area, such that at least some of the fibres of one bunch come into contact with the fibres of another bunch. At least some of the fibres of a bunch may be held on the scalp without contact with the adhesive, by entanglement with the fibres of this bunch and by contact with the fibres of the other bunch.

Before applying the fibres of a bunch, forces may be applied to the fibres of this bunch in order to entangle or to increase the entanglement of the fibres. For example, it is possible to roll the bunch between the fingers in the same way as a ball might be rolled between the fingers.

Bunch

The bunch preferably comprises between 50 and 1000 fibres, better still between 100 and 600 fibres.

At least half of the fibres of the bunch, better still at least 75% of the fibres of the bunch, even better at least 90%, or even all of the fibres of the bunch, have a radius of curvature over at least part of their length of between 1 and 10 mm, preferably between 1 and 5 mm. The length of the fibres of the bunch, when straightened, is preferably between 1 and 100 mm, better still between 3 and 80 mm, even better between 5 and 40 mm, the standard deviation of the distribution of lengths of the fibres of the bunch being preferably such that σ/m>20%, better still σ/m>30%.

The average diameter of the fibres of the bunch is preferably between 40 and 120 μm. Preferably, said bunch comprises curly fibres, at least 50% of the fibres being preferably curly, even better at least 75% of the fibres being curly, or even all of the fibres being curly. “Curly fibre” is understood to be a fibre whose longitudinal axis describes at least one revolution about a winding axis.

The bunch may include fibres of circular and/or non-circular cross-section.

The bunch may be in the general form of a ball before application.

A single bunch may comprise, where appropriate, fibres of length less than 5 mm and fibres of length greater than 5 mm, in particular up to 40 mm.

A single bunch may comprise only fibres of length greater than 5 mm.

The fibres of the bunch may be black or any other colour, in particular identical to that of the existing hair. A bunch may comprise a mixture of fibres of different colours.

The fibres may be formed of natural or synthetic fibres, in particular of thermoplastic material.

A bunch may comprise a single type of fibres or alternatively a mixture of fibres of different colours, diameters, lengths and/or natures.

Where appropriate, the bunches of fibres are produced at least partly from the hair of the person on whose scalp the fibres are to be glued. For example, a bunch of fibres is formed by mixing fibres obtained by cutting the hair where it is found in abundance with longer and/or shorter synthetic fibres, in particular of substantially the same colour, which help to make the bunch of fibres cohesive.

At rest, when it is picked up by the user in order to apply it, the bunch of fibres may occupy a total volume of between a sphere of diameter 5 mm and a concentric sphere of diameter 50 mm.

Assembly

Another subject of the invention, according to another of its aspects, is an assembly for performing the method according to the invention as defined above, comprising:
at least one bunch of at least 50 entangled fibres, said bunch comprising fibres having a radius of curvature of between 1 and 10 mm over at least part of their length,
an adhesive to be applied to an area of the scalp.

The fibres and the adhesive are preferably packed in the same package.

The bunch may present some or all of the features mentioned above. The entanglement of the fibres of the bunch enables the bunch to be manipulated between two fingers, in order to tap the fibres of this bunch against an area of scalp covered with adhesive, as explained above. Thus, the entanglement of the fibres is such that the fibres remain attached to the bunch while it is being gripped, until it is applied.

### BRIEF DESCRIPTION OF THE DRAWINGS

The invention may be better understood from reading the following detailed description of non-limiting embodiments thereof and from examining the attached drawing, in which:

FIG. 1 shows an example of an assembly according to the invention,

FIG. 2 shows in isolation a bunch of fibres before it is applied to the scalp,

FIG. 3 illustrates the use of a bunch,

FIG. 4 shows an alopecic area before treatment, and

FIG. 5 shows the area after treatment.

### DETAILED DESCRIPTION

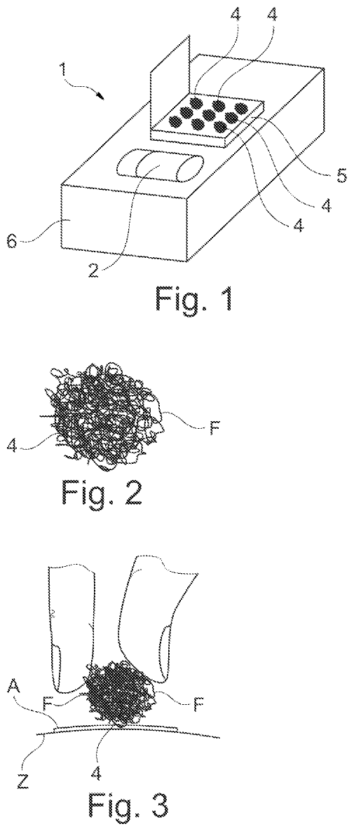

FIG. 1 shows an example of an assembly 1 for performing the method according to the invention.

This assembly 1 comprises a cosmetic adhesive A for application to the scalp, contained in a container 2. Said container may be of any type and may include any means of application, such as a fine brush or an applicator end piece; the adhesive may also be applied by spraying. The adhesive A is chosen for example from polymers, in particular those having a Tg<20° C. and preferably <−20° C., in particular from polyacrylates or polymethacrylates or copolymers containing acrylate units or alkyl methacrylate with a carbon chain greater than C4.

The adhesive may also be a non-polymeric compound, such as a wax with a melting point <40° C.

The polymer or wax may be dissolved in an appropriate solvent and in particular a volatile solvent (the boiling point of which is preferentially <190° C. and preferably <140° C.).

The polymer or wax may also be in a dispersed form, in other words in the state of microscopic particles in a solvent that is incapable of dissolving said polymer or wax. In particular, the polymer or wax may be dispersed in water or in a volatile organic solvent such as isododecane.

The polymers or waxes preferably have a mass concentration of >20%.

The assembly 1 also comprises bunches 4 of fibres, which may or may not be packaged separately. Each bunch 4 is in the general form of a ball, for example, as shown in FIG. 2. In the example shown, the bunches 4 are packaged in bulk in a box 5.

The container 2 and the box 5 may be sold together to the user in a common package 6, of the blister pack or case type, for example.

Each bunch 4 is formed by a bundle of entangled fibres F, which retains its cohesion when manipulated by hand, for example by being held between two fingers. In other words, the fibres of a bunch 4 are sufficiently in contact with one another that they are not separated when handled.

The fibres of each bunch 4 are chosen so as to produce a result that blends with the hair remaining in or adjacent to the area to be filled.

For each bunch, fibres having the following features are preferably used:

1) Length: The average “extended” length of the fibres is between 1 and 100 mm, preferably between 3 and 80 mm, better still between 5 and 40 mm; the standard deviation of the distribution of lengths of the fibres in a bunch is preferably such that σ/m >20%, better still >30%.
2) Diameter: The average diameter of the fibres is between 40 and 120 μm.
3) Cross-section: The cross-section of a fibre may be any shape: non-circular, in particular twisted elliptical or elliptical, or circular.
4) Curvature: The radius of curvature of at least a portion of the length of the fibres is between 1 and 10 mm, for example between 1 and 5 mm.

With such features, a density and distribution of fibres close to the natural density is obtained, and application is simple.

The material of the fibres may be chosen from natural or synthetic materials. As natural materials, keratin fibres, fibres based on cellulose or modified cellulose, such as cotton, viscose or linen, may be chosen. As synthetic materials, polyamides (Nylon), PBT, PE, PP, acrylic fibres (modacrylic) or polyester fibres may be chosen.

The fibres may be entangled during fabrication, by tossing, for example.

The cosmetic treatment according to the invention is performed as follows.

The adhesive A is applied to a number of areas Z of the scalp on which it is desired to deposit the fibres F in order to recreate the impression of the presence of a head of hair at this point or to give the impression of a denser head of hair. The adhesive A is applied in such a way that the skin has an adhesive appearance. The film of adhesive formed on the skin may be continuous or discontinuous.

Then, the bunches 4 are grasped one by one between the thumb and the index finger, in order to apply them to the adhesive A, as shown in FIG. 3. In this way, the fibres are brought into contact with the adhesive as a group rather than individually.

The fibres F are applied by touches to the adhesive. The application is performed by tapping successively on the area to be treated. Often, the area to be treated is larger in size than the diameter of the bunch, and so the bunch is moved over the adhesive surface until either there are no more fibres in the bunch or the area to be treated is covered with fibres in an aesthetic manner. In the case of a small bunch, all the fibres may be applied in one go, where appropriate.

Optionally, the treatment may be finished by rubbing the area Z with the fibres deposited thereon in order to homogenise or improve the contact between the fibres and the adhesive.

The fibres F are preferably brought into contact with the adhesive A in such a way that they are only partially in contact with the adhesive, for example over at most 20% of their length. The application of a bunch of entangled fibres in a generally spherical form ensures that when the fibres are brought into contact with the adhesive, there is a minority of fibres for which the plane with the curve is parallel to the plane formed by the adhesive film. The risk of fibres having too great a contact length is reduced in this way.

This advantageously ensures that not all of the fibres are in contact with the adhesive, benefiting from the entanglement of the fibres to hold the fibres that are not attached directly to the skin by the adhesive. An SL ratio as defined above of less than 12% can be achieved in this way. Furthermore, this approach enables fibres from one bunch to help to hold the fibres from another bunch.

If necessary, to improve the hold of the fibres when they are handled, it is possible to manipulate the bunch of fibres between the fingers, by pressing and rolling the ball of fibres between the fingers, which tends to reinforce their entanglement.

Such a manipulation is particularly useful for bunches 4 composed of fibres of length greater than 5 mm.

In the case of shorter fibres, of length less than or equal to 5 mm, the grip can be improved by integrating longer fibres into the bunch or by combining several bunches, namely a bunch of short fibres and a bunch of longer fibres, by mixing them, in order to form a more cohesive bunch.

### Example

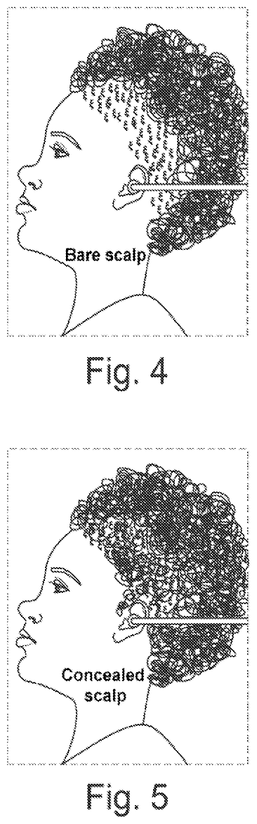

A film of PROS-AIDE Cream Blend 331 Adhesive is applied to an area Z of the scalp, as shown in FIG. 4. A fine brush, a coarse brush, a sponge, a flocked applicator or a siliconised applicator may be used.

After a few minutes, the film reaches its maximum tack.

At this precise moment, bunches of fibres are brought into contact with the adhesive. The fibres are brought into contact with the adhesive by tapping the bunch against the adhesive present on the area to be treated, as shown in FIG. 3. It is easy to control the quantity of fibres deposited, by repeating the operation in the same area.

The result shown in FIG. 5 is obtained.

The invention is not limited to the embodiments described above. For example, a bunch of fibres may be applied by holding said bunch with an applicator. Just part of the bunch of fibres may be grasped, using tweezers. These may be direct-action tweezers, i.e. applied by means of the user's hand, the user controlling the gripping force, especially when the only remaining fibres are in the pinch area. In another configuration they may be reverse-action tweezers, i.e. a spring applies the gripping force.

The applicator may equally be in the form of a pad, the transfer area of which is slightly adhesive. Bringing this adhesive area into contact with a bunch of fibres enables the bunch to be handled and moved to the treatment area. The adhesive capacity of the applicator is lower than the adhesive capacity of the glue applied to the treatment area.

1. A cosmetic treatment method comprising:
(a) applying an adhesive to an area of the scalp,
(b) applying fibres to said area covered with adhesive, belonging to a bunch of at least 50 entangled fibres, said bunch comprising fibres having, at least over part of their length, a radius of curvature of between 1 and 10 mm, the application taking place in such a way that for at least some of the fibres of the bunch, they only come into contact with the adhesive over part of their length.

2. The method according to claim 1, wherein said bunch comprising between 50 and 1000 fibres.

3. The method according to claim 1, wherein at least half of the fibres of the bunch having a radius of curvature over at least part of their length of between 1 and 5 mm.

4. The method according to claim 1, wherein the length of the fibres of the bunch, when straightened, being between 1 and 100 mm, the standard deviation being such that σ/m>20%.

5. The method according to claim 1, wherein the average diameter of the fibres of the bunch being between 40 and 120 μm.

6. The method according to claim 1, wherein the application of the fibres to said area being performed manually by tapping the area covered with adhesive with the bunch held between two fingers.

7. The method according to claim 1, wherein said bunch comprising curly fibres.

8. The method according to claim 1, wherein the bunch comprising fibres of circular cross-section.

9. The method according to claim 1, wherein the bunch comprising fibres of non-circular cross-section.

10. The method according to claim 1, wherein for at least half the fibres of the bunch, they only come into contact with the adhesive over at most 20% of their length.

11. The method according to claim 1, wherein some of the fibres of the bunch remaining attached to the fibres already deposited, by entanglement therewith, without coming into contact with the adhesive.

12. The method according to claim 11, wherein with SL between 6 and 12%, where SL is the ratio between, firstly, the sum of the lengths of fibres deposited with a portion thereof in contact with the adhesive and, secondly, the sum of the lengths of all the fibres of the bunch.

13. The method according to claim 1, further comprising the application of fibres from at least two bunches to said area, such that at least some of the fibres of one bunch come into contact with the fibres of another bunch.

14. The method according to claim 11, wherein at least some of the fibres of a bunch being held on the scalp without contact with the adhesive by entanglement with the fibres of this bunch and by contact with the fibres of the other bunch.

15. The method according to claim 1, wherein the bunch is in the general form of a ball before its application.

16. The method according to claim 1, wherein the bunch comprising fibres of length less than 5 mm and fibres of length greater than 5 mm.

17. An assembly for performing the method as defined in claim 1, comprising:
at least one bunch of at least 50 entangled fibres, said bunch comprising fibres having a radius of curvature of between 1 and 10 mm, over at least part of their length,
an adhesive to be applied to an area of the scalp.

18. The assembly according to claim 17, wherein the fibres and the adhesive being packed in the same package.

19. The assembly according to claim 17, further comprising a bunch as defined in claim 2.

20. The assembly according to claim 17, wherein all the fibres of the bunch being of length greater than 5 mm.
